(19) United States
(12) Patent Application Publication
(10) Pub. No.: US 2022/0369949 A1
(43) Pub. Date: Nov. 24, 2022

(54) METHOD AND SYSTEM FOR DETECTING CONCENTRATION OF ANALYTE BASED ON CHANGE IN RELATIVE PERMITTIVITY OF BIOLOGICAL TISSUE WITHIN LIVING BODY

(71) Applicant: SB Solutions Inc., Ulsan (KR)

(72) Inventors: Namhwan Sung, Ulsan (KR); Jagannath Malik, Ulsan (KR)

(73) Assignee: SB Solutions Inc., Ulsan (KR)

(21) Appl. No.: 17/399,812

(22) Filed: Aug. 11, 2021

(30) Foreign Application Priority Data
May 18, 2021 (KR) 10-2021-0063882

(51) Int. Cl. A61B 5/055; G01N 27/22; G01N 33/483; A61B 5/145; A61B 5/00

(52) U.S. Cl. CPC A61B 5/055; G01N 27/221; G01N 27/228; G01N 33/4833; A61B 5/14532; A61B 5/725; A61B 5/7278; A61B 5/7203

(57) ABSTRACT

Disclosed are a method and system for detecting a concentration of an analyte based on a change in relative permittivity of a biological tissue within a living body. The method of detecting a concentration of an analyte may include generating a fringing field, measuring a change in a resonant frequency generated by an oscillator based on a change in capacitance attributable to a change in an analyte within a region of the fringing field, and measuring a change characteristic of the analyte within the fringing field based on the change in the resonant frequency.

### CROSS-REFERENCES TO RELATED APPLICATIONS

This application is based on and claims priority under 35 U.S.C. 119 to Korean Patent Application No. 10-2021-0063882, filed on May 18, 2021, in the Korean intellectual property office, the disclosures of which are herein incorporated by reference in their entireties.

### TECHNICAL FIELD

The following description relates to a method and system for detecting a concentration of an analyte based on a change in relative permittivity of a biological tissue within a living body.

### BACKGROUND OF THE INVENTION

Examples in which adult-onset diseases, such as diabetes, hyperlipidemia and thrombosis, are increased are continuously reported. Such diseases need to be periodically measured using various bio sensors because it is important to continuously monitor and manage the diseases. A common type of bio sensor is a method of injecting, into a test strip, blood drawn from a finger and then quantizing an output signal by using an electrochemical method or a photometry method. Such an approach method causes a user a lot of pain because blood needs to be drawn every time.

For example, in order to manage diabetes of hundreds of millions of people around the globe, the most basic thing is to measure blood glucose. Accordingly, a blood glucose measurement device is an important diagnostic unit essential for a diabetes patient. Various blood glucose measurement devices are recently developed, but the most common method is a method of gathering blood by pricking a patient's finger and directly measuring a concentration of glucose within the blood. An invasive method includes a method of penetrating an invasive sensor into the skin, measuring a concentration of glucose through the invasive sensor for a given time, and measuring blood glucose by recognizing the blood glucose through an external reader.

In contrast, a non-invasive method includes a method using a light-emitting diode (LED)-photo diode (PD). However, the non-invasive method has low accuracy due to environmental elements and foreign substances, such as sweat or a temperature, because the LED-PD is attached to the skin.

The aforementioned information is to merely help understanding, and may include contents which do not form a part of a conventional technology and may not include contents which may be presented to those skilled in the art through a conventional technology.

[Prior Art Document Number]
Korean Patent No. 10-2185556

### SUMMARY

This summary is provided to introduce a selection of concepts in a simplified form that are further described below in the Detailed Description. This summary is not intended to identify key characteristics of the claimed subject matter, nor is it intended to be used as an aid in determining the scope of the claimed subject matter.

The present disclosure provides a method and system for detecting a concentration of an analyte, which can detect a concentration of an analyte by detecting a change in capacitance attributable to a change in an analyte within the region of a fringing field and detecting and measuring a change in the resonant frequency generated by an oscillator based on a change in the capacitance.

In one aspect, there is provided a method of detecting a concentration of an analyte, including generating a fringing field, measuring a change in a resonant frequency generated by an oscillator based on a change in capacitance attributable to a change in an analyte within a region of the fringing field, and measuring a change characteristic of the analyte within the fringing field based on the change in the resonant frequency.

According to an aspect, generating the fringing field may include generating the fringing field by using a fringing field capacitor included in the oscillator.

According to another aspect, measuring the change in the resonant frequency generated by the oscillator may include generating a periodic oscillation signal by using a feedback network including a fringing field capacitor included in the oscillator and a frequency selection filter and configured to feed a part of an output signal back as an input in order to provide a required phase shift.

According to yet another aspect, measuring the change characteristic of the analyte may include measuring a change in capacitance attributable to a change in permittivity by using a material under test (MUT) unit including a material having a dielectric constant.

According to yet another aspect, the method may further include measuring a temperature by using a temperature sensor and incorporating the measured temperature into the change in the resonant frequency or the measured change characteristic.

According to yet another aspect, the method may further include obtaining activity information of an object based on an angular speed measured by a gyro sensor and compensating for the measured change characteristic based on the obtained activity information of the object.

In another aspect, there is provided a system for detecting a concentration of an analyte, including a sensor unit configured to measure a change characteristic of an analyte. The sensor unit is configured to generate a fringing field, measure a change in a resonant frequency generated by an oscillator based on a change in capacitance attributable to a change in the analyte within a region of the fringing field, and measure a change characteristic of the analyte within the fringing field based on the change in the resonant frequency.

According to an aspect, the sensor unit may include the oscillator configured to generate the fringing field and generate a periodic oscillation signal.

According to another aspect, the oscillator may include a fringing field capacitor configured to generate the fringing field and a feedback network configured to comprise a frequency selection filter and to feed a part of an output signal back as an input in order to provide a required phase shift.

According to yet another aspect, the fringing field capacitor may include a sensor pattern configured to generate the fringing field and a material under test (MUT) unit including a material having a dielectric constant and configured to measure a change in capacitance attributable to a change in permittivity.

According to yet another aspect, the system may further include a band pass filter (BPF) configured to filter out a signal, having a frequency out of filter specifications, from an output signal of the sensor, a buffer configured to provide matching between an output of the BPF and an input of a counter in order to prevent a signal loss, and the counter configured to count a frequency of a scalation signal as a zero-cross detection circuit for a signal output by the buffer.

According to yet another aspect, the system may further include a temperature sensor configured to measure a temperature around the sensor unit. The sensor unit may incorporate the measured temperature into the change in the resonant frequency or the measured change characteristic.

According to yet another aspect, the system may further include a processing unit configured to control an operation of the sensor unit, a communication unit configured to communicate with another external device, an output unit configured to output at least one of a concentration of the analyte calculated based on the change characteristic of the analyte and a warning based on the concentration of the analyte, and a power management unit configured to provide power to at least one of the processing unit, the communication unit and the output unit.

According to yet another aspect, the power management unit may include at least one of a battery and a component for wireless power transmission, and the component may include a circuit and antenna for wireless power transmission.

According to yet another aspect, the system may further include a noise removal unit configured to remove noise from a signal output by the sensor unit.

According to yet another aspect, the system may further include a gyro sensor configured to measure an angular speed of the system. The measured change characteristic may be compensated for based on activity information of an object obtained based on the angular speed.

A concentration of an analyte can be detected by detecting a change in capacitance attributable to a change in the analyte within the region of a fringing field and detecting and measuring a change in the resonant frequency of a frequency generated by the oscillator based on a change in capacitance.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a block diagram illustrating an example of internal components of a system for detecting a concentration of an analyte according to an embodiment of the present disclosure.

FIG. 2 is a diagram illustrating an example of an oscillator according to an embodiment of the present disclosure.

FIG. 3 is a diagram illustrating another example of internal components of the system for detecting a concentration of an analyte according to an embodiment of the present disclosure.

FIG. 4 is a diagram illustrating an example of a sensor according to an embodiment of the present disclosure.

FIG. 5 is a diagram illustrating an example in which a fringing field is generated in an embodiment of the present disclosure.

FIG. 6 is a diagram illustrating an example of interactions between a biological tissue, an interstitial fluid (ISF), a blood vessel, and a fringing field depending on a penetration depth of the fringing field in an embodiment of the present disclosure.

FIG. 7 is an example illustrating the intensity of an electromagnetic field and a degree of skin penetration according to a sensor pattern in an embodiment of the present disclosure.

FIG. 8 is a diagram illustrating an example of the system for detecting a concentration of an analyte, which has a wrist watch type, in an embodiment of the present disclosure.

FIG. 9 is a graph illustrating blood glucose levels continuously measured for each frequency outputted by the sensor and for each time zone in an embodiment of the present disclosure.

FIG. 10 is a diagram illustrating an example in which a change or a rate of change in an analyte is calibrated based on a temperature in an embodiment of the present disclosure.

FIG. 11 is a flowchart illustrating an example of a method of detecting a concentration of an analyte according to an embodiment of the present disclosure.

### DETAILED DESCRIPTION

While illustrative embodiments have been illustrated and described, it will be appreciated that various changes can be made therein without departing from the spirit and scope of the invention.

Hereinafter, embodiments are described in detail with reference to the accompanying drawings. However, the embodiments may be changed in various ways, and the scope of right of this patent application is not limited or restricted by such embodiments. It is to be understood that all changes, equivalents and substitutions of the embodiments are included in the scope of right.

Terms used in embodiments are merely used for a description purpose and should not be interpreted as intending to restrict the present disclosure. An expression of the singular number includes an expression of the plural number unless clearly defined otherwise in the context. In this specification, it should be understood that a term, such as “include” or “have”, is intended to designate the presence of a characteristic, a number, a step, an operation, a component, a part or a combination of them described in the specification, and does not exclude the existence or possible addition of one or more other characteristics, numbers, steps, operations, components, parts, or combinations of them in advance.

All terms used herein, including technical or scientific terms, have the same meanings as those commonly understood by a person having ordinary knowledge in the art to which an embodiment pertains, unless defined otherwise in the specification. Terms, such as those commonly used and defined in dictionaries, should be construed as having the same meanings as those in the context of a related technology, and are not construed as being ideal or excessive unless explicitly defined otherwise in the specification.

Furthermore, in describing the present disclosure with reference to the accompanying drawings, the same component is assigned the same reference numeral regardless of its reference numeral, and a redundant description thereof is omitted. In describing an embodiment, a detailed description of a related known art will be omitted if it is deemed to make the gist of the embodiment unnecessarily vague.

Furthermore, in describing components of an embodiments, terms, such as a first, a second, A, B, (a), and (b), may be used. Such terms are used only to distinguish one component from the other component, and the essence, order, or sequence of a corresponding component is not limited by the terms. When it is said that one component is “connected”, “combined”, or “coupled” to the other component, the one component may be directly connected or coupled to the other component, but it should also be understood that a third component may be “connected”, “combined”, or “coupled” between the two components.

A component included in any one embodiment and a component including a common function are described using the same name in another embodiment. Unless described otherwise, a description written in any one embodiment may be applied to another embodiment, and a detailed description in a redundant range is omitted.

A system for detecting a concentration of an analyte according to an embodiment may give a warning to a user as an emergency alarm when a value of a biological component predicted by an algorithm based on an immediately measured value or a measured value is greater than a risk level of the biological component.

Such a system for detecting a concentration of an analyte may be implemented in a smart watch type or an embedded type, but the present disclosure is not limited thereto. For example, the system for detecting a concentration of an analyte may be implemented in the form of an implant device inserted into the body of an object. The smart watch type may have a structure including a sensor and a measurement circuit closely attached to a body around the wrist. The embedded type has a structure including a measurement circuit, and a sensor within a band may be placed in an upper arm. In some embodiments, the sensor may be inserted into one or both sides of the band.

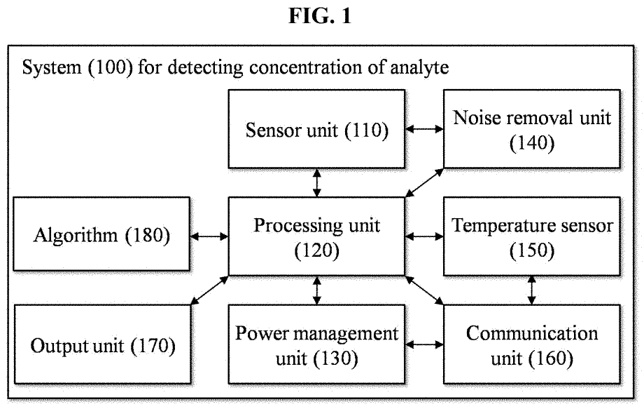

FIG. 1 is a block diagram illustrating an example of internal components of a system for detecting a concentration of an analyte according to an embodiment of the present disclosure. The system 100 for detecting a concentration of an analyte according to the present disclosure may include a sensor unit 110, a processing unit 120, a power management unit 130, a noise removal unit 140, a temperature sensor 150, a communication unit 160 and an output unit 170 as hardware devices. Furthermore, the system 100 for detecting a concentration of an analyte may further include an algorithm 180 which may be used to process data measured by the sensor unit 110.

In this case, the output of the sensor unit 110 may correspond to a concentration of one or more analytes present within a bio sample. A fringing field from the sensor unit 110 may be used to detect a fine change in a bio permittivity level attributable to the presence of one or more analytes. The sensor unit 110 may incorporate a change in the permittivity level into a change in the oscillation frequency. As a more detailed example, a sensor may measure a change in the resonant frequency generated by an oscillator based on a change in capacitance attributable to a change in an analyte within the region of the fringing field, and may detect a concentration of the analyte by measuring a change characteristic of the analyte within the fringing field (or a change in the concentration) based on a change in the resonant frequency.

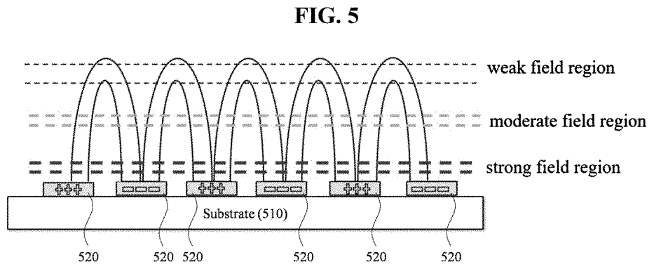

The fringing field may be formed by an electromagnetic force line between two conductors when a voltage is biased to a capacitor, for example. Such a fringing field is more specifically described later with reference to FIG. 5.

The processing unit 120 may include a micro controller unit (MCU), and may control operations of the sensor unit 110, the noise removal unit 140, the temperature sensor 150, the communication unit 160 and/or the output unit 170. For example, the processing unit 120 may control operations of the sensor unit 110, the noise removal unit 140, the temperature sensor 150, the communication unit 160 and/or the output unit 170 according to the algorithm 160.

The power management unit 130 may provide power to the sensor unit 110, the processing unit 120, the noise removal unit 140, the temperature sensor 150, the communication unit 150 and/or the output unit 170. The power management unit 130 may include a battery and/or a circuit and antenna for wireless power transmission.

The noise removal unit 140 may be implemented to remove noise from a signal output by the sensor unit 110. For example, the noise removal unit 140 may be implemented to remove high frequency noise. For another example, since a capacitance value may vary due to a heartbeat, the noise removal unit 140 may be implemented to calibrate noise attributable to the heartbeat.

The temperature sensor 150 may be implemented to measure a temperature around the system 100 for detecting a concentration of an analyte. The temperature may also affect a biological permittivity level. The processing unit 120 may determine an oscillation frequency of the oscillator attributable to a biological permittivity level based on the original sensor frequency outputted by the sensor unit 110 and temperature data outputted by the temperature sensor 150. For example, the processing unit 120 may calibrate an original sensor frequency outputted by the sensor unit 110 based on a temperature, and may calculate a rate of change in an analyte level in sensing data (i.e., a change in the oscillation frequency (or resonant frequency) based on a change in capacitance) measured based on the calibrated original sensor frequency. Substantially, the processing unit 120 may determine a change or a rate of change in an analyte by incorporating a temperature, measured by the temperature sensor 150, into a change in the resonant frequency or a change characteristic measured with respect to the analyte.

The communication unit 160 may include a wired and/or wireless communication device for enabling the system 100 for detecting a concentration of an analyte to communicate with another device. Communication between the system 100 for detecting a concentration of an analyte and another device through the communication unit 160 may be performed using Bluetooth low energy (BLE), WiFi and/or a 5-th generation mobile communication technology (5G), but the present disclosure is not limited thereto.

The output unit 170 may output a visual, auditory and/or tactile signal to the outside of the system 100 for detecting a concentration of an analyte. For example, the output unit 170 may include a light-emitting diode (LED), a beeper and/or a vibrator in order to provide a user with a warning based on an analyte level. In some embodiments, the output unit 170 may include a digital display device for displaying a measured analyte level.

The sensor unit 110 may include an oscillator used to generate a periodic oscillation signal in an electronic circuit. The oscillator may generate a periodic waveform by only DC power supply. An output waveform may have a square wave, a sine wave or a non-sine wave depending on the type of oscillator. The oscillator according to an embodiment may include a sine wave oscillator having feedback. The feedback oscillator may consist of a transistor and/or an amplifier (e.g., an operational amplifier (OP-AMP)), a capacitor (or fringing field capacitor), a register network, a feedback component and/or a gain control circuit/component. The feedback is a process of some of an output signal being fed back as an input in order to adjust an additional output.

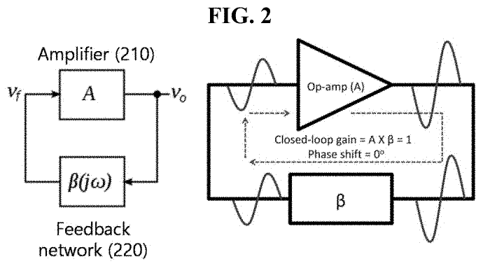

FIG. 2 is a diagram illustrating an example of an oscillator according to an embodiment of the present disclosure. FIG. 2 illustrates an example in which some of an output signal outputted through the amplifier 210 is fed back as an input to the amplifier 210 through a feedback network 220 in order to adjust an additional output. The feedback network includes a frequency selection filter, and may provide a required phase shift. For example, a circuit for the feedback network 220 may be implemented as an RC or LC component, but a 3-stage RC network may be preferably used as the feedback network 220. FIG. 2 illustrates a phase shift as 0°, but this is merely one example, and the present disclosure is not limited thereto.

The oscillator may be classified depending on a frequency selection filter used in the feedback network 220. The RC oscillator is a sort of feedback oscillator in which a filter consists of a network of a resistor R and a capacitor C.

In this case, the capacitor used in the oscillator may be commonly a fringing field capacitor that generates a fringing field. For example, an inter-digitized electrode type capacitor may be used as the capacitor.

The oscillator may be basically used to generate a low frequency in a sub-MHz frequency range. An RC oscillator consisting of an RC network, which may be used to generate a phase shift necessary for a response signal as described above, may be used as the oscillator. The RC network may be used to achieve positive feedback that generates an oscillating sine wave voltage. Such an oscillator has excellent frequency intensity, low noise and jitter.

When power is supplied to the circuit, a noise voltage starts to oscillate. The RC network shifts the phase of an output signal by 180° and then supplies the signal to the circuit again as an input, so that continuous oscillation occurs in the circuit.

The LC oscillator consists of an inductor L and a capacitor C, and may form a tank circuit. Such an oscillator is suitable for high-frequency oscillation, but may not achieve required inductance having a small form factor in a low frequency. Accordingly, currently, an RC oscillator may be denoted in the oscillator used in the sensor unit 110, but the use of the LC oscillator is not excluded.

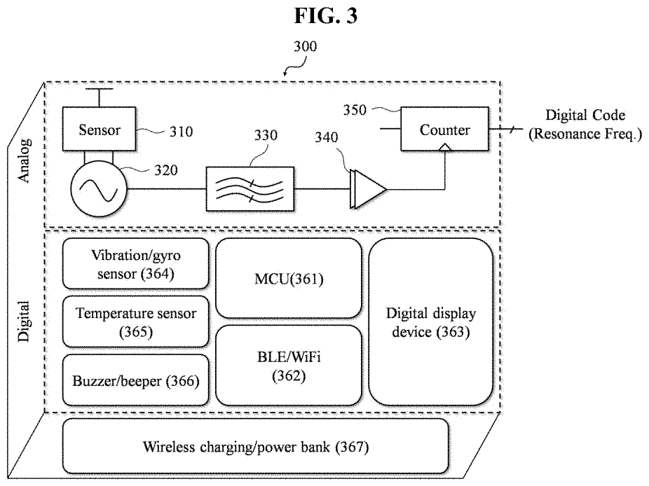

FIG. 3 is a diagram illustrating another example of internal components of the system for detecting a concentration of an analyte according to an embodiment of the present disclosure. The system 300 for detecting a concentration of an analyte according to the present disclosure may include a sensor 310, an oscillator 320, a band pass filter (BPF) 330, a buffer 340, and a counter 350 as analog components. Such analog components may be included in the sensor unit 110 described with reference to FIG. 1.

The sensor 310 may be implemented in a form to substantially include a fringing field capacitor included in the oscillator 320. The fringing field capacitor may form a fringing field. As a change in capacitance attributable to a change in an analyte within the region of the fringing field is incorporated into the oscillator 320, an oscillation frequency (or resonant frequency) generated by the oscillator 320 may be changed. At this time, the system 300 for detecting a concentration of an analyte may measure a change characteristic of the analyte within the fringing field (e.g., a change in the concentration of the analyte) based on a change in the resonant frequency.

The BPF 330 is a frequency selection filter that passes a signal having a specific bandwidth. A signal having a frequency out of filter specifications (e.g., a frequency lower than a filter-low cutoff frequency and higher than a filter-high cutoff frequency) may be filtered out at the output of the BPF 330.

The buffer 340 may be used to provide input-output matching between two different circuit components. This is a kind of electric impedance conversion from one circuit to the other circuit, and can prevent a signal loss. For example, the buffer 340 may provide matching between the output of the BPF 330 and the input of the counter 350.

The counter 350 is a circuit for counting the frequency of a scalation signal, and may include a zero-cross detection circuit for an input signal in common.

The system 300 for detecting a concentration of an analyte may include an MCU 361, BLE/WiFi 362, a digital display device 363, a vibration/gyro sensor 364, a temperature sensor 365, and buzzer/beeper 366 as digital components. In this case, the MCU 361 may correspond to or may be included in the processing unit 120 described with reference to FIG. 1. Furthermore, the BLE/WiFi 362 may correspond to or may be included in the communication unit 160 described with reference to FIG. 1. Furthermore, the temperature sensor 365 may correspond to or may be included in the temperature sensor 150 described with reference to FIG. 1. The digital display device 363, the vibration/gyro sensor 364 and the buzzer/beeper 366 may be included in the output unit 170 described with reference to FIG. 1. Although separately described, the gyro sensor may be used to obtain activity information of an object. The gyro sensor is not included in the output unit 170, and may operate as a separate sensor such as the temperature sensor 365, may measure an angular speed of the system 300 for detecting a concentration of an analyte, and may obtain activity information of an object based on the measured angular speed. The obtained activity information may be used to compensate for a concentration of an analyte measured by the sensor unit 110 or to determine the validity of the measured concentration of the analyte. For example, when a change in a value of activity information is equal to or greater than a threshold, the MCU 361 may request the sensor unit 110 to measure a concentration of an analyte again. For another example, when a change in a value of activity information is equal to or greater than the threshold, the MCU 361 may compensate for a measured change characteristic.

Furthermore, the system 300 for detecting a concentration of an analyte may further include a wireless charging/power bank 367. In this case, the “wireless charging” may mean a circuit and antenna for wireless power transmission, and the “power bank” may mean a battery. In other words, the wireless charging/power bank 367 may correspond to or may be included in the power management unit 130 described with reference to FIG. 1.

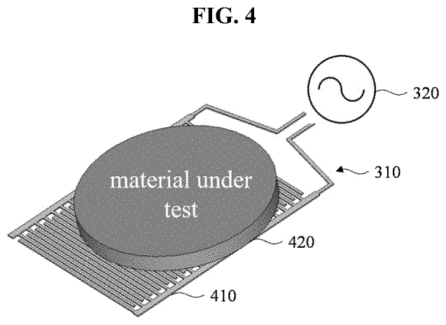

FIG. 4 is a diagram illustrating an example of the sensor according to an embodiment of the present disclosure. FIG. 4 illustrates the sensor 310 and the oscillator 320 described with reference to FIG. 3. As described above, the sensor 310 may be implemented in a form to substantially include a capacitor included in the oscillator 320. In FIG. 4, a sensor pattern 410 may correspond to such a capacitor, and may play a role to generate a fringing field. The sensor 310 may further include a material under test (MUT) unit 420. In this case, the sensor pattern 410 may generate a fringing field. The MUT unit 420 is made of a material having a dielectric constant, and may measure a change in capacitance attributable to a change in permittivity. The MUT unit 420 may be disposed at the center location of the sensor 310 because the sensor 310 commonly operates sensitively at the center location.

FIG. 5 is a diagram illustrating an example in which a fringing field is generated in an embodiment of the present disclosure. FIG. 5 illustrates an example in which a fringing field is formed on a substrate 510 as an oscillation signal is applied to metallic sensor traces 520 as the sensor pattern 410 described with reference to FIG. 4. In this case, FIG. 5 illustrates a strong field region, a moderate field region and a weak field region as the fringing field. That is, FIG. 5 illustrates that the intensity of the fringing field is weakened as the distance from the metallic sensor traces 520 becomes distant.

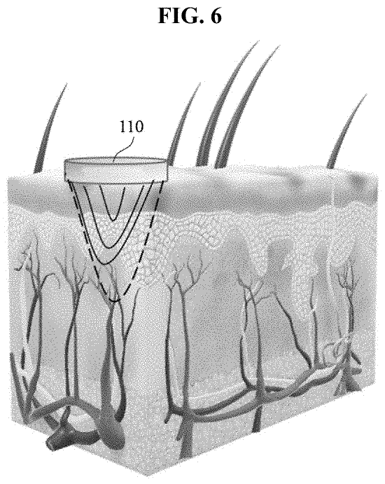

FIG. 6 is a diagram illustrating an example of interactions between a biological tissue, an interstitial fluid (ISF), a blood vessel, and a fringing field depending on a penetration depth of the fringing field in an embodiment of the present disclosure. As described above, The fringing field from the sensor unit 110 may be used to detect a fine change in a bio permittivity level attributable to the presence of one or more analytes. The sensor unit 110 may incorporate a change in the permittivity level into a change in the oscillation frequency (or a change in the resonant frequency).

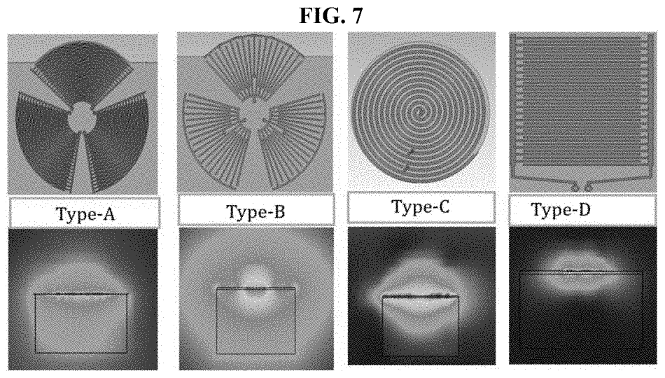

FIG. 7 is an example illustrating the intensity of an electromagnetic field and a degree of skin penetration according to a sensor pattern in an embodiment of the present disclosure. FIG. 7 illustrates that the intensity of an electromagnetic field and a degree of skin penetration are different depending on the type of sensor pattern included in the sensor 310. Accordingly, a concentration of various biological analytes can be detected by selectively using a sensor pattern depending on the type of biological analyte (a location within a living body depending on a type) to be measured.

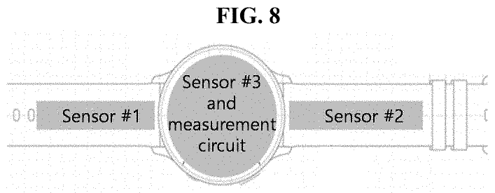

FIG. 8 is a diagram illustrating an example of the system for detecting a concentration of an analyte, which has a wrist watch type, in an embodiment of the present disclosure. The embodiment of FIG. 8 illustrates an example the system for detecting a concentration of an analyte includes a measurement circuit and three sensors. FIG. 8 is an implementation example of the system for detecting a concentration of an analyte, and illustrates an example having a wrist watch type. As described above, the system for detecting a concentration of an analyte may be implemented in an embedded type, and may also be implemented in the form of an implant device inserted into the body of an object. Furthermore, it may be easily understood that the number and locations of sensors may be variously changed, if necessary.

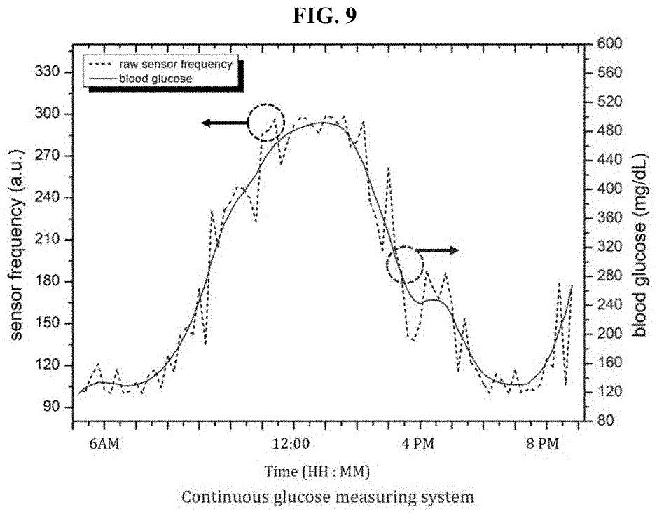

FIG. 9 is a graph illustrating blood glucose levels continuously measured for each frequency outputted by the sensor and for each time zone in an embodiment of the present disclosure. The graph illustrates sensor output frequencies and corresponding blood glucose levels. The sensor may provide a continuous frequency output according to immediate blood glucose levels. If necessary, high frequency noise and a change may be removed using a tracking, prediction or averaging filter. This may be implemented in both hardware and software algorithms.

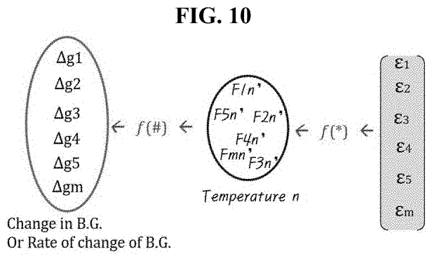

FIG. 10 is a diagram illustrating an example in which a change or a rate of change in an analyte is calibrated based on a temperature in an embodiment of the present disclosure. As described above, the sensor 310 may incorporate a change in permittivity into a change in the oscillation frequency. In this case, since the temperature also affects a permittivity level, the system 300 (or the MCU 361) for detecting a concentration of an analyte may calibrate an oscillation frequency of the oscillator attributable to a biological permittivity level through the temperature. The system 300 (or the MCU 361) for detecting a concentration of an analyte may determine a change or a rate of change in an analyte by using a mapping function f(*) and a calibration function f(#). For example, the system 300 (or the MCU 361) for detecting a concentration of an analyte may determine a change or a rate of change in an analyte into which a measured temperature has been incorporated by incorporating the temperature into a change in the resonant frequency or a change characteristic measured with respect to analyte.

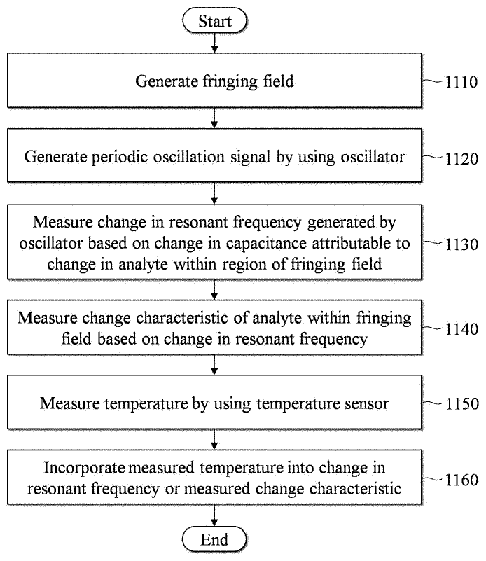

FIG. 11 is a flowchart illustrating an example of a method of detecting a concentration of an analyte according to an embodiment of the present disclosure. The method of detecting a concentration of an analyte according to the present disclosure may be performed by the system 100 for detecting a concentration of an analyte, described with reference to FIG. 1, for example.

In step 1110, the system 100 for detecting a concentration of an analyte may generate a fringing field. For example, the fringing field may be generated by the fringing field capacitor of the oscillator included in the sensor unit 110.

In step 1120, the system 100 for detecting a concentration of an analyte may generate a periodic oscillation signal by using an oscillator. For example, the oscillator may correspond to the oscillator 320 described with reference to FIG. 3. In this case, the system 100 for detecting a concentration of an analyte includes the fringing field capacitor included in the oscillator and the frequency selection filter, and may generate the periodic oscillation signal by using the feedback network for feeding a part of an output signal back as an input in order to provide a required phase shift.

In step 1130, the system 100 for detecting a concentration of an analyte may measure a change in a resonant frequency generated by the oscillator based on a change in capacitance attributable to a change in an analyte within the region of the fringing field. As described above, a fine change in a bio permittivity level attributable to the presence of one or more analytes in the fringing field may be detected. In this case, the sensor unit 110 may incorporate a change in the permittivity level into a change in the oscillation frequency (or a change in the resonant frequency). For example, the system 100 for detecting a concentration of an analyte may measure a change in capacitance attributable to a change in permittivity by using the MUT unit including a material having a dielectric constant.

In step 1140, the system 100 for detecting a concentration of an analyte may measure a change characteristic of the analyte within the fringing field based on a change in the resonant frequency. As described above, a change in a permittivity level may be incorporated into a change in the oscillation frequency (or a change in the resonant frequency). This may mean that a change in the analyte within the region of the fringing field has been incorporated into a change in the oscillation frequency. The system 100 for detecting a concentration of an analyte may measure a change characteristic of the analyte based on a change in the permittivity level, which has been incorporated into a change in the resonant frequency.

In step 1150, the system 100 for detecting a concentration of an analyte may measure a temperature by using the temperature sensor. The temperature sensor may be implemented to measure a temperature around the system 100 for detecting a concentration of an analyte or the sensor unit 110 included in the system 100 for detecting a concentration of an analyte, for example.

In step 1160, the system 100 for detecting a concentration of an analyte may incorporate the measured temperature into a change in the resonant frequency or the measured change characteristic. For example, in step 1160, the system 100 for detecting a concentration of an analyte may calculate a change in the resonant frequency into which the temperature has been incorporated or a change or a rate of change in a concentration of the analyte based on the change characteristic.

In some embodiments, the system 100 for detecting a concentration of an analyte may obtain activity information of an object based on an angular speed measured by the gyro sensor, and may compensate for a measured change characteristic based on the obtained activity information of the object. Furthermore, in some embodiments, the system 100 for detecting a concentration of an analyte may determine whether to measure a concentration of an analyte again based on the obtained activity information of the object.

As described above, according to the embodiments of the present disclosure, capacitance according to a change in an analyte within the region of a fringing field is changed. A concentration of the analyte can be detected by detecting, by the sensor, the changed capacitance and detecting and measuring a change in a resonant frequency generated by the oscillator.

The aforementioned system or device may be implemented as a hardware component, a software component and/or a combination of a hardware component and a software component. For example, the device and components described in the embodiments may be implemented using one or more general-purpose computers or special-purpose computers, for example, a processor, a controller, an arithmetic logic unit (ALU), a digital signal processor, a microcomputer, a field programmable gate array (FPGA), a programmable logic unit (PLU), a microprocessor or any other device capable of executing or responding to an instruction. A processing device may perform an operating system (OS) and one or more software applications executed on the OS. Furthermore, the processing device may access, store, manipulate, process and generate data in response to the execution of software. For convenience of understanding, one processing device has been illustrated as being used, but a person having ordinary knowledge in the art may understand that the processing device may include a plurality of processing components and/or a plurality of types of processing components. For example, the processing device may include a plurality of processors or one processor and one controller. Furthermore, other processing configurations, such as a parallel processor, are also possible.

Software may include a computer program, a code, an instruction or a combination of one or more of them, and may configure a processor so that it operates as desired or may instruct processors independently or collectively. Software and/or data may be embodied in any type of a machine, component, physical device, virtual equipment, or computer storage medium or device so as to be interpreted by the processor or to provide an instruction or data to the processor. The software may be distributed to computer systems connected over a network and may be stored or executed in a distributed manner. The software and data may be stored in one or more computer-readable recording media.

The method according to the embodiment may be implemented in the form of a program instruction executable by various computer means and stored in a computer-readable recording medium. The computer-readable recording medium may include a program instruction, a data file, and a data structure alone or in combination. The program instructions stored in the medium may be specially designed and constructed for the present disclosure, or may be known and available to those skilled in the field of computer software. Examples of the computer-readable storage medium include magnetic media such as a hard disk, a floppy disk and a magnetic tape, optical media such as a CD-ROM and a DVD, magneto-optical media such as a floptical disk, and hardware devices specially configured to store and execute program instructions such as a ROM, a RAM, and a flash memory. Examples of the program instructions include not only machine language code that is constructed by a compiler but also high-level language code that can be executed by a computer using an interpreter or the like.

As described above, although the embodiments have been described in connection with the limited embodiments and the drawings, those skilled in the art may modify and change the embodiments in various ways from the description. For example, proper results may be achieved although the aforementioned descriptions are performed in order different from that of the described method and/or the aforementioned components, such as the system, configuration, device, and circuit, are coupled or combined in a form different from that of the described method or replaced or substituted with other components or equivalents.

Accordingly, other implementations, other embodiments, and the equivalents of the claims fall within the scope of the claims.

1. A method of detecting a concentration of an analyte with the system of claim 7, comprising:
generating a fringing field;
measuring a change in a resonant frequency generated by an oscillator based on a change in capacitance attributable to a change in an analyte within a region of the fringing field; and
measuring a change characteristic of the analyte within the fringing field based on the change in the resonant frequency.

2. The method of claim 1, wherein generating the fringing field comprises generating the fringing field by using a fringing field capacitor included in the oscillator.

3. The method of claim 1, wherein measuring the change in the resonant frequency generated by the oscillator comprises generating a periodic oscillation signal by using a feedback network comprising a fringing field capacitor included in the oscillator and a frequency selection filter and configured to feed a part of an output signal back as an input in order to provide a required phase shift.

4. The method of claim 1, wherein measuring the change characteristic of the analyte comprises measuring a change in capacitance attributable to a change in permittivity by using a material under test (MUT) unit comprising a material having a dielectric constant.

5. The method of claim 1, further comprising:
measuring a temperature by using a temperature sensor; and
incorporating the measured temperature into the change in the resonant frequency or the measured change characteristic.

6. The method of claim 1, further comprising:
obtaining activity information of an object based on an angular speed measured by a gyro sensor; and
compensating for the measured change characteristic based on the obtained activity information of the object.

7. A system for detecting a concentration of an analyte, comprising:
a sensor unit configured to measure a change characteristic of an analyte,
wherein the sensor unit is configured to:
generate a fringing field,
measure a change in a resonant frequency generated by an oscillator based on a change in capacitance attributable to a change in the analyte within a region of the fringing field;
measure a change characteristic of the analyte within the fringing field based on the change in the resonant frequency; and
detect a concentration or a change in the concentration of the analyte from the measurement of the change characteristic,
wherein the sensor unit comprises metallic sensor traces arranged in a flattened pattern, wherein the metallic sensor traces are configured in pairs to carry opposite polarity, the pattern is selected from one of the following: pairs of spiral sensor traces, pairs of radially inward-extending and radially outward-extending sensor traces in multiple circular sectors, and pairs of sensor traces extending in arcs in multiple circular sectors, wherein each pattern produces a different intensity of electromagnetic field or degree of skin penetration, and wherein each pattern is selected for the analyte being measured.

8. The system of claim 7, wherein the sensor unit comprises the oscillator configured to generate the fringing field and generate a periodic oscillation signal.

9. The system of claim 8, wherein the oscillator comprises:
a fringing field capacitor configured to generate the fringing field; and
a feedback network configured to comprise a frequency selection filter and to feed a part of an output signal back as an input in order to provide a required phase shift.

10. The system of claim 8, wherein the fringing field capacitor comprises:
a sensor pattern configured to generate the fringing field; and
a material under test (MUT) unit comprising a material having a dielectric constant and configured to measure a change in capacitance attributable to a change in permittivity.

11. The system of claim 8, further comprising:
a band pass filter (BPF) configured to filter out a signal, having a frequency out of filter specifications, from an output signal of the sensor;
a buffer configured to provide matching between an output of the BPF and an input of a counter in order to prevent a signal loss; and
the counter configured to count a frequency of a signal as a zero-cross detection circuit for a signal output by the buffer.

12. The system of claim 7, further comprising a temperature sensor configured to measure a temperature around the sensor unit,
wherein the sensor unit incorporates the measured temperature into the change in the resonant frequency or the measured change characteristic.

13. The system of claim 7, further comprising:
a processing unit configured to control an operation of the sensor unit;
a communication unit configured to communicate with another external device;
an output unit configured to output at least one of a concentration of the analyte calculated based on the change characteristic of the analyte and a warning based on the concentration of the analyte; and
a power management unit configured to provide power to at least one of the processing unit, the communication unit and the output unit.

14. The system of claim 13, wherein:
the power management unit comprises at least one of a battery and a component for wireless power transmission, and
the component comprises a circuit and antenna for wireless power transmission.

15. The system of claim 7, further comprising a filter configured to remove noise from a signal output by the sensor unit.

16. The system of claim 7, further comprising a gyro sensor configured to measure an angular speed of the system,
wherein the measured change characteristic is compensated for based on activity information of an object obtained based on the angular speed.
